(19) United States
(12) Patent Application Publication
(10) Pub. No.: US 2026/0199266 A1
(43) Pub. Date: Jul. 16, 2026

(54) VANILLIN HYDRAZONE COMPOUNDS

(71) Applicant: Renovel Innovations, Inc., Fremont, CA (US)

(72) Inventors: Bishwajit Nag, Union City, CA (US); Ananda Sen, Castro Valley, CA (US); Nitish Nag, Union City, CA (US); Srinivasan Narasimahan, Chennai (IN); Rupali Agarwal, Fremont, CA (US)

(21) Appl. No.: 19/448,613

(22) Filed: Jan. 14, 2026

Related U.S. Application Data
(60) Provisional application No. 63/745,173, filed on Jan. 14, 2025.

(51) Int. Cl. A61K 31/15; A61K 31/216; A61K 31/455; A61P 3/04; A61P 3/06; A61P 3/10; C07C 69/92; C07C 249/16; C07C 309/76; C07D 213/55

(52) U.S. Cl. CPC A61K 31/15; A61K 31/216; A61K 31/455; A61P 3/04; A61P 3/06; A61P 3/10; C07C 69/92; C07C 249/16; C07C 309/76; C07D 213/55

(57) ABSTRACT

Novel Vanillin Hydrazone compounds are provided which exhibit activity for the treatment of immunological diseases, inflammation, obesity, hyperlipidemia, hypertension, neurological diseases and diabetes

### FIELD OF THE INVENTION

The present invention relates to novel vanillin hydrazone derivatives for the treatment of immunological diseases, inflammation, obesity, hyperlipidemia, hypertension, neurological diseases and diabetes

### BACKGROUND OF INVENTION

Metabolic syndrome, Insulin resistance syndrome or Syndrome X is a name for a group of risk factors that occur together and increase the risk for coronary artery disease, stroke, and type 2 diabetes. Metabolic syndrome is becoming more and more common globally specially in the United States. Researchers are not sure whether the syndrome is due to one single cause, but all of the risks for the syndrome are related to obesity. The two most important risk factors for metabolic syndrome are: Extra weight around the middle and upper parts of the body (central obesity) and insulin resistance. The body uses insulin less effectively than normal. Insulin is needed to help control the amount of sugar in the body. As a result, blood sugar and fat levels rise. Other risk factors include Aging, Genes, Hormone changes, Lack of exercise. People who have metabolic syndrome often have two other problems that can either cause the condition or make it worse. Excess blood clotting, and increased levels of blood substances that are a sign of inflammation throughout the body

Metabolic syndrome is affiliated with three or more of the following signs: Blood pressure equal to or higher than 130/85 mmHg, Fasting blood sugar (glucose) equal to or higher than 100 mg/dL, Large waist circumference (length around the waist Men—40 inches or more and Women—35 inches or more, Low HDL cholesterol (Men—under 40 mg/dL Women—under 50 mg/dL) and Triglycerides equal to or higher than 150 mg/dL. In general, metabolic syndrome is a combination of Type 2 Diabetes, Obesity, Hyperlipidemia and hypertension

People with metabolic syndrome have an increased long-term risk for developing heart disease, type 2 diabetes, stroke, kidney disease, and poor blood supply to the legs. There is no one single treatment option available to treat metabolic syndrome. Current drugs that control blood glucose are usually not effective in lowering body weight, hypertension and cholesterol. Similarly, drugs that manage lipid levels may or may not have impact on other metabolic parameters. The present invention was aimed to develop new class of therapeutics derived, modified and chemically synthesized from natural product which can combat multiple arms of metabolic syndrome. The invention also describes one such core group of molecules with synthesis scheme and biological data for diabetes, obesity, inflammation, hypertension and Hyperlipidemia.

### SUMMARY OF INVENTION

The present invention relates to novel Vanillin Hydrazone derivatives of the formula I (see FIG. 1) where the general structure has been segregated into two parts, A and B, wherein, Part A is always a vanillin moiety with an aryloxy functional group attached to the oxygen of the hydroxyl group, where X is aryl or pyridyl. Part B is an alkyl or aryl hydrazine, the aryl group of which is substituted by a nitro, di nitro, cyano groups and the alkyl group is either branched or linear chain with any number of carbons of the general formula CH3(CH2)n-CO—NHNH2 where n=2, 3 . . . 18. The patent of compound 1 includes their derivatives, their analogs, their tautomeric forms, their stereoisomers, their polymorphs, their pharmaceutically acceptable salts, their pharmaceutically acceptable solvates, wherein X corresponds to substituted or unsubstituted pyridyl or aryl acid chloride.

The present invention also relates to a process for the preparation of the above said novel compounds, their analogs, their tautomeric forms, their stereoisomers, their polymorphs, their pharmaceutically acceptable salts, their pharmaceutically acceptable solvates, novel intermediates and pharmaceutical composites containing them. Tautomeric forms are isomeric forms which exists in a state of equilibrium capable of reacting according to either form. Stereoisomers include configurational isomers, such as cis- and trans double bonds, as well as optically active isomers having different spatial arrangements of their atoms. Polymorphs are molecules which can crystallize in two or more forms. Solvates are molecular or ionic complexes of molecules or ions of solvent with those of a solute. The amino acid derivatives are included, but not limited to naturally occurring amino acids. Analogs include those compounds which differ by substitution of an oxygen, sulphur, nitrogen or carbon atom in place of such an atom. Analogs also include atoms of the same family of the Periodic Table, such as F, Cl, Br and I. Derivatives include compounds resulting from routine functionalizing of atoms, such as, derivatives found by protecting amino or carboxyl groups by carboxylation or esterification, respectively.

### BRIEF DESCRIPTION OF FIGURES

FIG. 1 shows the formula I with the general structure used in the patent.

FIG. 2 shows the various R1, R2, R3, X, R4, R5, R6, R7, R8 representations and IUPAC names for each of the compounds.

FIG. 3 depicts the process for the preparation of the compounds represented by formula I.

FIG. 4 shows that at the end of 90 days, the group of mice treated with RNV-235 (Compound 1) showed a decrease by 28% in body weight compared with the Control group on high fat diet.

FIG. 5 shows that the group of mice administered with RNV-235 (Compound 1) showed an 8% decrease in fasting blood glucose at 60 days compared with the control mice on high fat diet group (FIG. 5A). An oral glucose tolerance test was conducted at 60 days, for which the mice were administered with glucose orally at 2 g/Kg and blood glucose was measured at the fasted state (baseline/taken prior to glucose administration), then at 30, 60, 90 and 120 minutes (FIG. 5B). The group of mice treated with RNV-235 (Compound 1) showed a 10% decrease in blood glucose compared at the peak oral glucose tolerance test of 30 minutes at 60 days compared with the Control high fat diet group (FIG. 5C).

FIG. 6 shows that the group of mice administered with RNV-235 (Compound 1) showed a 10% decrease in fasting blood glucose at 90 days compared with the control mice on high fat diet group (FIG. 6A). An oral glucose tolerance test was conducted at 90 days, for which the mice were administered with glucose orally at 2 g/Kg and blood glucose was measured at the fasted state (baseline/taken prior to glucose administration), then at 30, 60, 90 and 120 minutes (FIG. 6B). The group of mice treated with RNV-235 (Compound 1) showed a significant decrease (P<0.001) by 37% in blood glucose compared at the peak oral glucose tolerance test of 30 minutes at 90 days compared with the Control high fat diet group (FIG. 6C).

FIG. 7 shows that Syrian Golden Hamsters when treated orally with RNV-235 (Compound 1) at 50 mg/kg for 60 days showed an 18% decrease in body weight when compared to the untreated cholesterol fed control.

FIG. 8 shows that the Syrian Golden Hamsters administered with RNV-235 (Compound 1) at 50 mg/kg for 60 days showed a significant decrease (P<0.0002) by 44% in Total Cholesterol (FIG. 8A) and 24% decrease in Triglycerides (FIG. 8B), compared with the Control high cholesterol group.

FIG. 9 shows the HDL level (FIG. 9A) and the LDL level (FIG. 9B) of male Syrian Golden Hamsters administered with RNV-235 (Compound 1) at 50 mg/kg for 60 days.

FIG. 10 shows blood glucose in male Syrian Golden Hamsters treated with RNV-235 (Compound 1) at 50 mg/kg, p.o. for 60 days.

FIG. 11 shows that at the end of 90 days, the group of mice treated with RNV-235 (Compound 1) showed a decrease by 34% in body weight compared with the Control high fat diet group.

FIG. 12 shows the Fasting blood glucose of SW mice on high fat diet treated with RNV-235 (Compound 1) at 0, 30, 60 and 90 days.

FIG. 13A shows the liver weight of SW mice on high fat diet treated with RNV-235 (Compound 1) after 90 days of treatment. FIG. 13B shows 22% decrease in the white adipose tissue weight of SW mice on high fat diet treated with RNV-235 (Compound 1) after 90 days of treatment.

FIG. 14 shows that at the end of 90 days, the group of mice treated with RNV-236 (Compound 2) showed a significant decrease (P<0.0001) by 43% in body weight compared with the Control high fat diet group.

FIG. 15 shows that the group of mice administered with RNV-236 (Compound 2) showed an 28% decrease in fasting blood glucose at 60 days compared with the control mice on high fat diet group (FIG. 15A). An oral glucose tolerance test was conducted at 60 days, for which the mice were administered with glucose orally at 2 g/Kg and blood glucose was measured at the fasted state (baseline/taken prior to glucose administration), then at 30, 60, 90 and 120 minutes (FIG. 15B). The group of mice treated with RNV-236 (Compound 2) showed a 17% decrease in blood glucose compared at the peak oral glucose tolerance test of 30 minutes at 60 days compared with the Control high fat diet group (FIG. 15C).

FIG. 16 shows that the group of mice administered with RNV-236 (Compound 2) showed a 8% decrease in fasting blood glucose at 90 days compared with the control mice on high fat diet group (FIG. 16A). An oral glucose tolerance test was conducted at 90 days, for which the mice were administered with glucose orally at 2 g/Kg and blood glucose was measured at the fasted state (baseline/taken prior to glucose administration), then at 30, 60, 90 and 120 minutes (FIG. 16B). The group of mice treated with RNV-236 (Compound 2) showed a significant decrease (P<0.002) by 28% in blood glucose compared at the peak oral glucose tolerance test of 30 minutes at 90 days compared with the Control high fat diet group (FIG. 16C).

FIG. 17 shows the effect of RNV-236 (Compound 2) at 50 mg/kg for 60 days on Syrian Golden Hamsters.

FIG. 18 shows that the Syrian Golden Hamsters administered with RNV-236 (Compound 2) at 50 mg/kg for 60 days showed a significant decrease by 20% in Total Cholesterol, compared with the Control high cholesterol group.

FIG. 19 shows the Triglycerides (FIG. 19A) and blood glucose (FIG. 19B) in male Syrian Golden Hamsters treated with RNV-236 (Compound 2) at 50 mg/kg for 60 days.

FIG. 20 shows that at the end of 90 days, the group of mice treated with RNV-237 (Compound 3) showed a significant (P<0.03) decrease by 31% in body weight compared with the Control high fat diet group.

FIG. 21 An oral glucose tolerance test was conducted at 60 days, for which the mice were administered with glucose orally at 2 g/Kg and blood glucose was measured at the fasted state (baseline/taken prior to glucose administration), then at 30, 60, 90 and 120 minutes (FIG. 21A). The group of mice treated with RNV-237 (Compound 3) showed a 10% decrease in blood glucose compared at the peak oral glucose tolerance test of 30 minutes at 60 days compared with the Control high fat diet group (FIG. 21B).

FIG. 22 shows that the group of mice administered with RNV-237 (Compound 3) showed a 5% decrease in fasting blood glucose at 90 days compared with the control mice on high fat diet group (FIG. 22A). An oral glucose tolerance test was conducted at 90 days, for which the mice were administered with glucose orally at 2 g/Kg and blood glucose was measured at the fasted state (baseline/taken prior to glucose administration), then at 30, 60, 90 and 120 minutes (FIG. 22B). The group of mice treated with RNV-237 (Compound 3) showed a significant decrease (P<0.01) by 22% in blood glucose compared at the peak oral glucose tolerance test of 30 minutes at 90 days compared with the Control high fat diet group (FIG. 22C).

FIG. 23 shows the effect of RNV-237 (Compound 3) on the body weight of a group of mice at the end of 90 days.

FIG. 24 shows the Fasting blood glucose of SW mice on high fat diet treated with RNV-237 (Compound 3) at 0, 30, 60 and 90 days.

FIG. 25A shows the liver weight (FIG. 25A) and white adipose tissue weight (FIG. 25B) of SW mice on high fat diet treated with RNV-237 (Compound 3) after 90 days of treatment.

FIG. 26 shows that the group of C57BL/6 mice treated orally with RNV-242 (Compound 4) at 50 mg/kg for 60 days followed by 30 days of no treatment showed a decrease by 12% in body weight.

FIG. 27 shows that the group of C57BL/6 mice administered with RNV-242 (Compound 4) showed a decrease in fasting blood glucose at 60 days compared with the control mice on high fat diet group (FIG. 27A). An oral glucose tolerance test was conducted at 60 days, for which the mice were administered with glucose orally at 2 g/Kg and blood glucose was measured at the fasted state (baseline/taken prior to glucose administration), then at 30, 60, 90 and 120 minutes (FIG. 27B). The group of mice treated with RNV-242 (Compound 4) showed a decrease in blood glucose compared at the peak oral glucose tolerance test of 30 minutes at 60 days compared with the Control high fat diet group (FIG. 27C).

FIG. 28 shows that the group of C57BL/6 mice administered with RNV-242 (Compound 4) showed a decrease in fasting blood glucose compared with the control group on high fat diet.

### DETAILED DESCRIPTION OF THE INVENTION

In an embodiment of the present invention, the group represented as X is derived from substituted or unsubstituted pyridyl or aryl acid chloride.

In an embodiment of the present invention, the groups represented as R1 to R8 are selected from Hydrogen, Halogens, hydroxyl, alkoxy, straight chain or branched alkyl, nitro, cyano and amino groups.

In an embodiment of the present invention, pharmaceutically acceptable salts forming part of this invention include base addition salts such as alkali metal salts like Li, Na, and K salts, alkaline earth metal salts like Ca and Mg salts, salts of organic bases such as lysine, arginine, guanidine, diethanolamine, chlorine and the like, ammonium or substituted ammonium salts. Salts may include acid addition salts which are sulphates, nitrates, phosphates, perchlorates, borates, hydrohalides, acetates, tartarates, maleates, citrates, succinates, palmoates, methanesulphonates, benzoates, ascorbates, glycerophosphates, ketoglutarates and the like. Pharmaceutically acceptable solvates may be hydrates or comprising other solvents of crystallization such as alcohols.

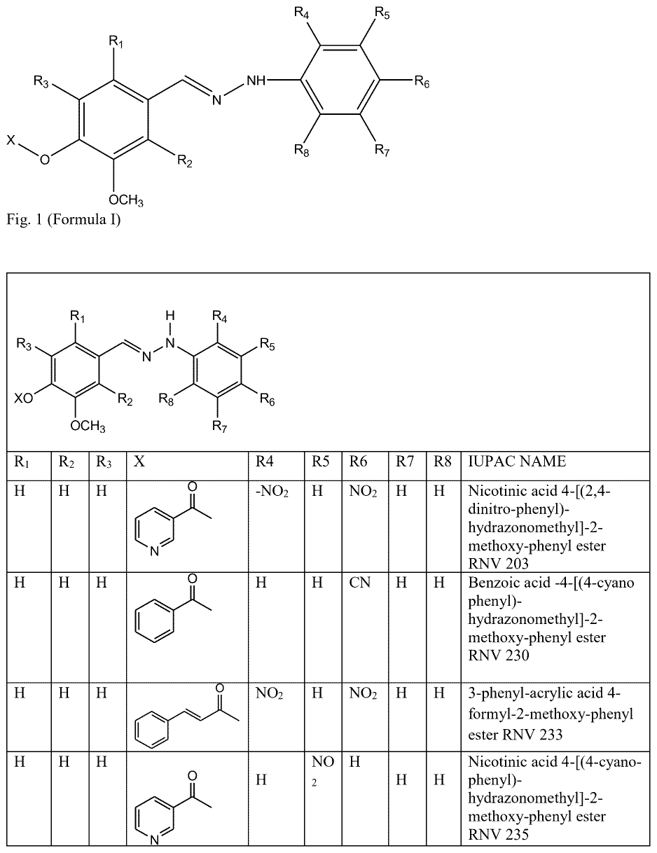

More preferably, the present innovation relates to novel Vanillin Hydrazone of formula I (see FIG. 1), their derivatives, their analogs, their tautomeric forms, their stereoisomers, their polymorphs, their pharmaceutically acceptable salts, their pharmaceutically acceptable solvates, novel intermediates and pharmaceutical composites containing them, wherein Part A, the vanillin ring has substituents R1, R2 and R3 which can be selected from Hydrogen, Halogens, hydroxyl, alkoxy, straight chain or branched alkyl, nitro, cyano and amino groups, and X, which can be selected from substituted or unsubstituted pyridyl or aryl acid chloride, and Part B having substituents R4, R5, R6, R7 and R8 which are selected from Hydrogen, Halogens, hydroxyl, alkoxy, straight chain or branched alkyl, nitro, cyano and amino groups.

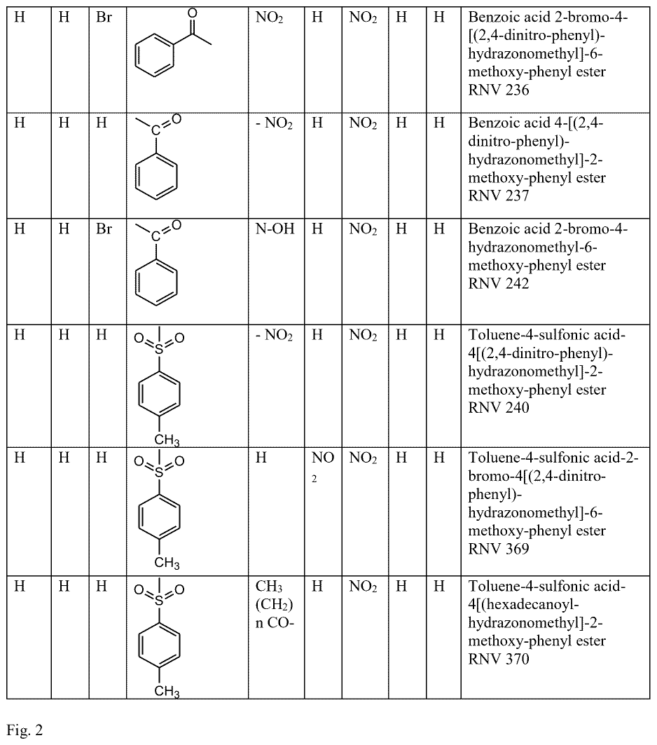

The formula of the useful compounds synthesized in this present are listed below. See FIG. 2 for the R1, R2, R3, X, R4, R5, R6, R7, R8 representations and IUPAC names for these compounds.

Nicotinic acid 4-[(2,4-dinitro-phenyl)-hydrazonomethyl]-2-methoxy-phenyl ester (RNV-203)

Benzoic acid-4-[(4-cyano phenyl)-hydrazonomethyl]-2-methoxy-phenyl ester (RNV-230)

3-phenyl-acrylic acid 4-formyl-2-methoxy-phenyl ester (RNV-233)

Nicotinic acid 4-[(4-cyano-phenyl)-hydrazonomethyl]-2-methoxy-phenyl ester (RNV-235)

Benzoic acid 2-bromo-4-[(2,4-dinitro-phenyl)-hydrazonomethyl]-6-methoxy-phenyl ester (RNV 236)

Benzoic acid 4-[(2,4-dinitro-phenyl)-hydrazonomethyl]-2-methoxy-phenyl ester (RNV-237)

Benzoic acid 2-bromo-4-hydrazonomethyl-6-methoxy-phenyl ester (RNV-242)

Toluene-4-sulfonic acid-4[(2,4-dinitro-phenyl)-hydrazonomethyl]-2-methoxy-phenyl ester (RNV-240)

Toluene-4-sulfonic acid-2-bromo-4[(2,4-dinitro-phenyl)-hydrazonomethyl]-6-methoxy-phenyl ester (RNV-369)

Toluene-4-sulfonic acid-4[(hexadecanoyl-hydrazonomethyl]-2-methoxy-phenyl ester (RNV-370)

Preferred salts for the compounds listed above are hydrochloride, hydrobromide, sodium, potassium or magnesium.

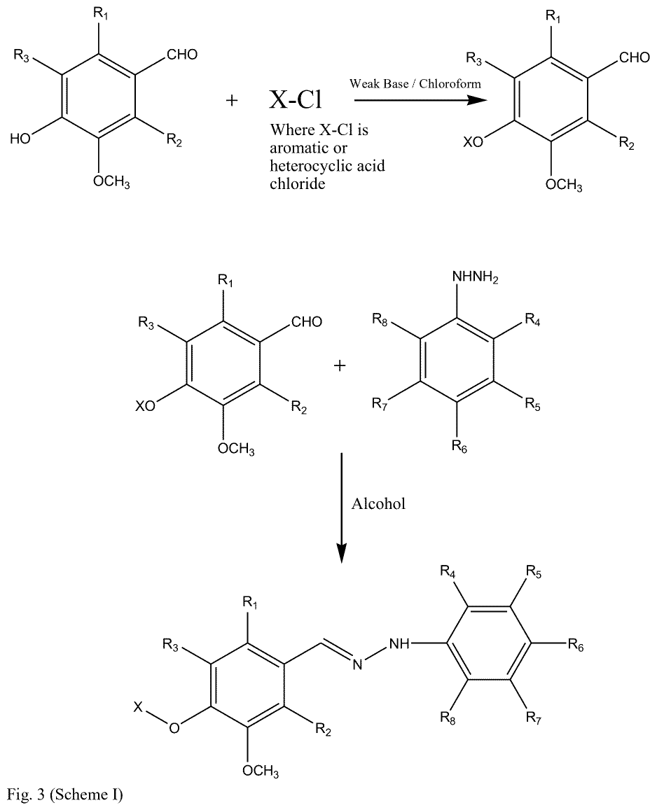

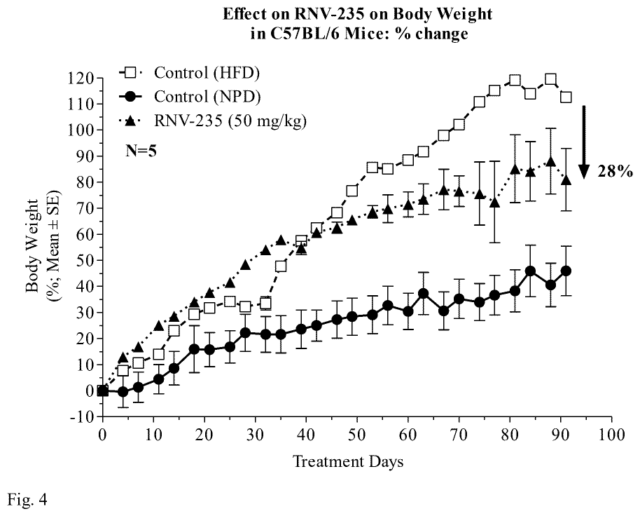

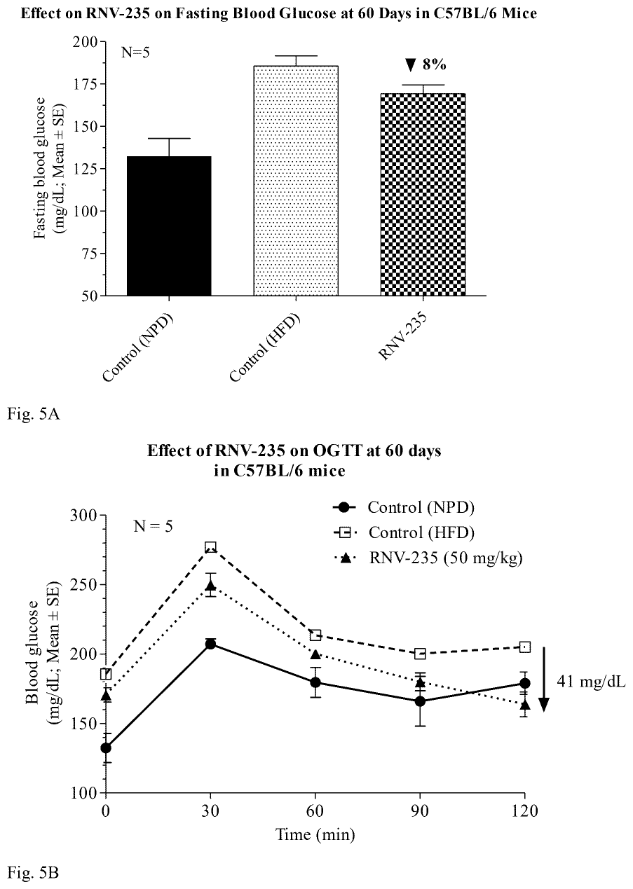

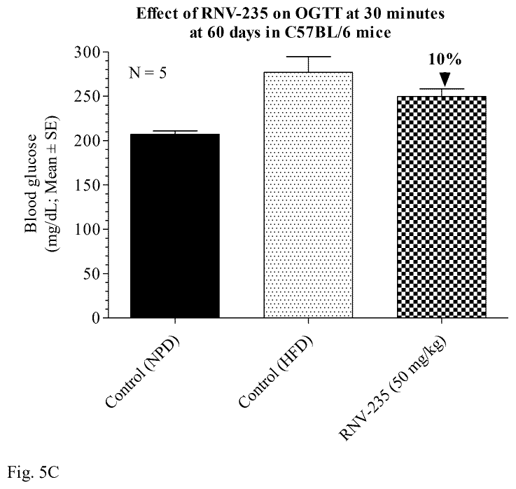

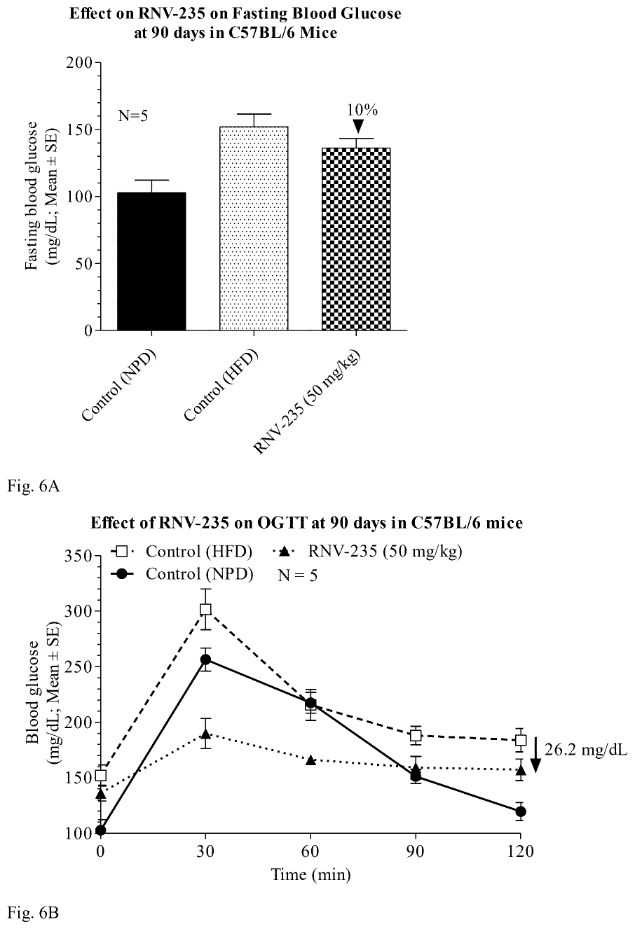

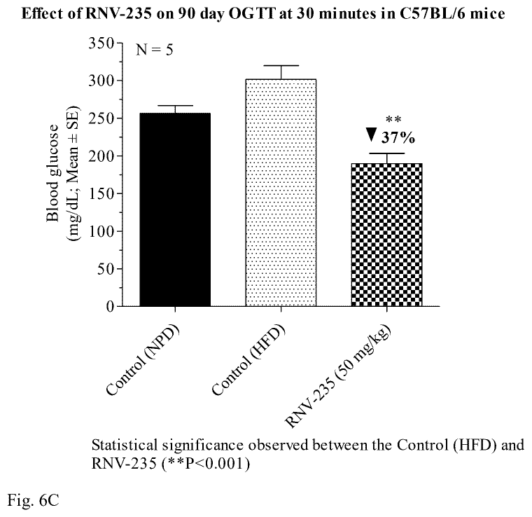

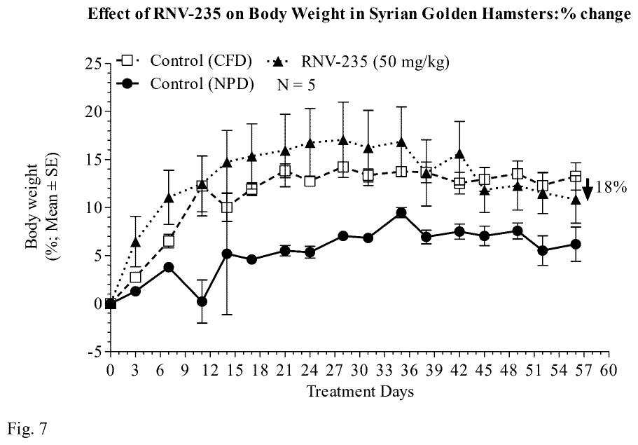

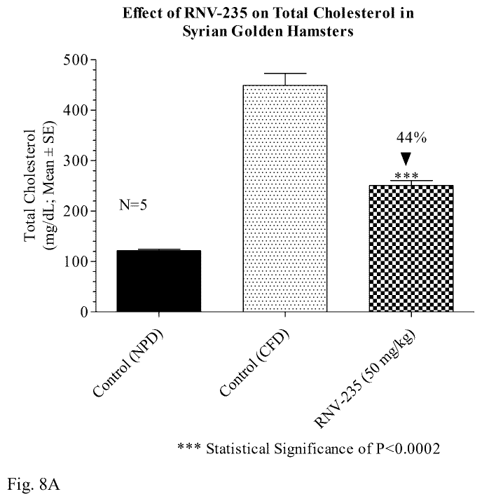

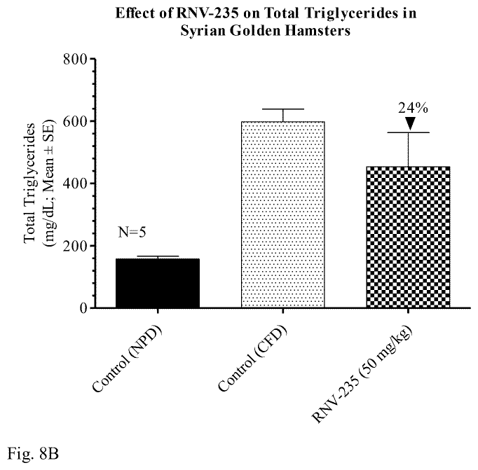

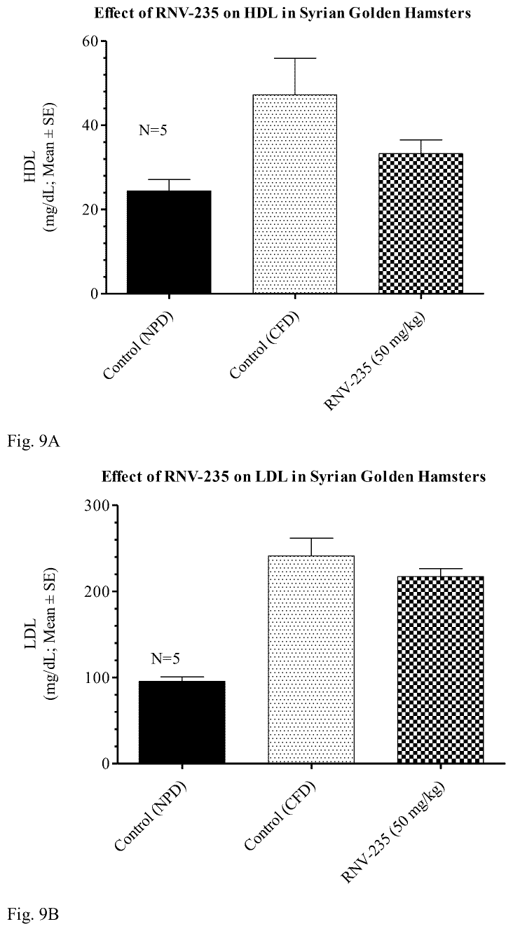

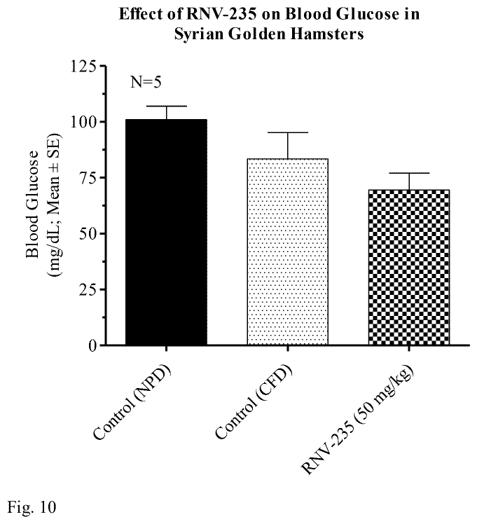

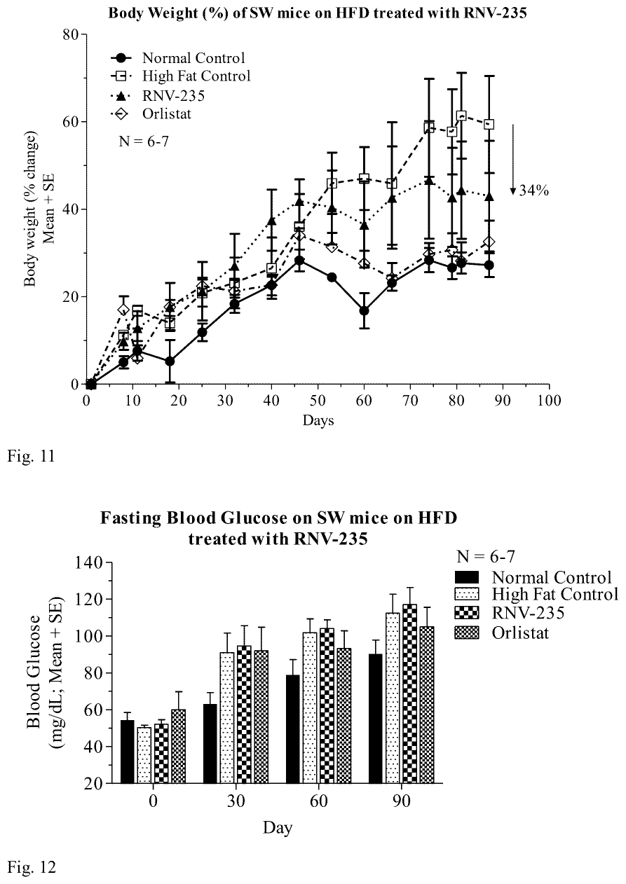

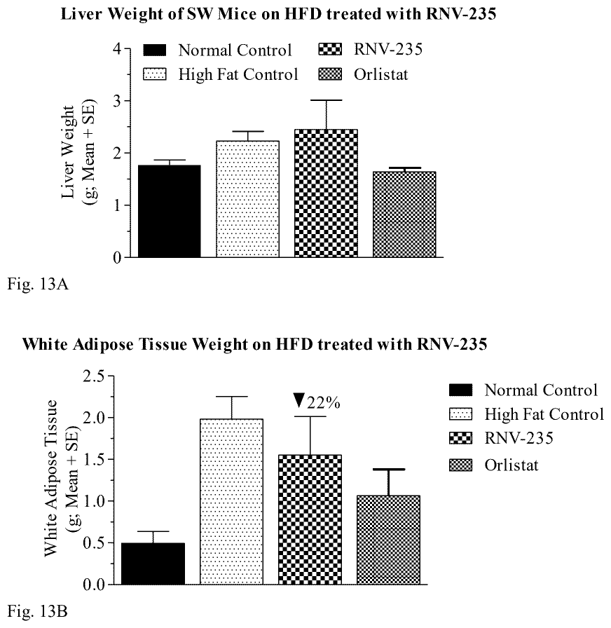

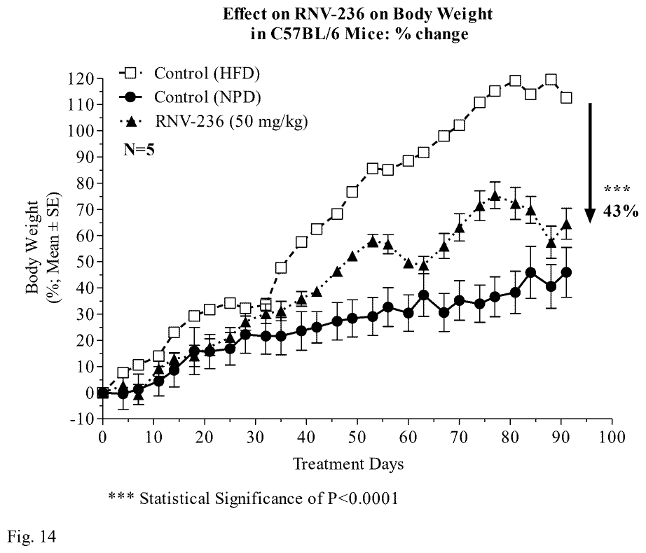

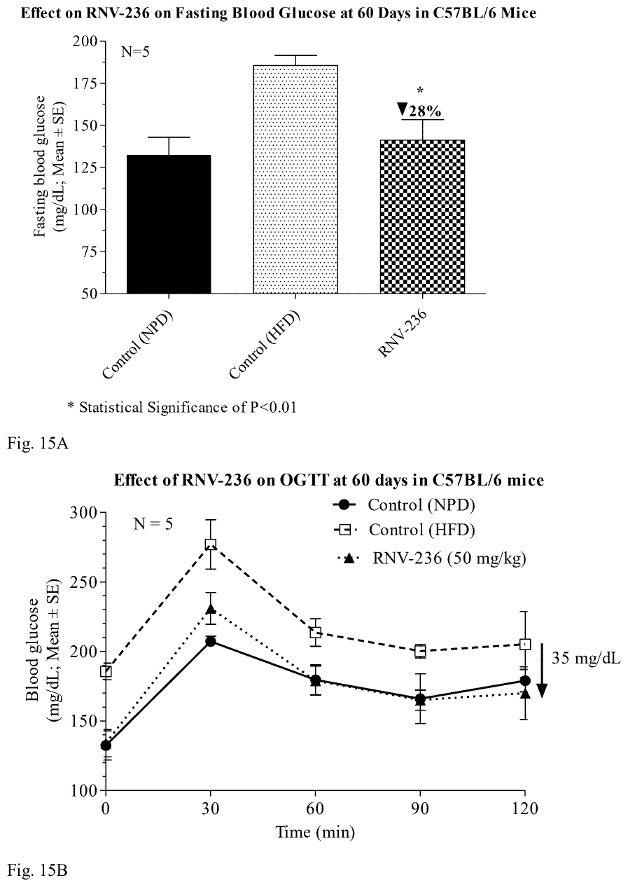

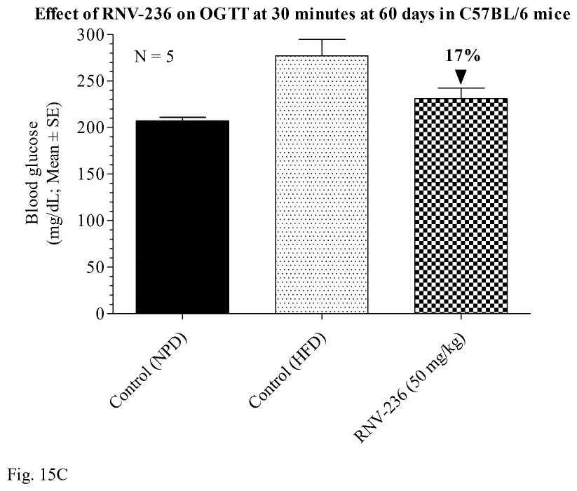

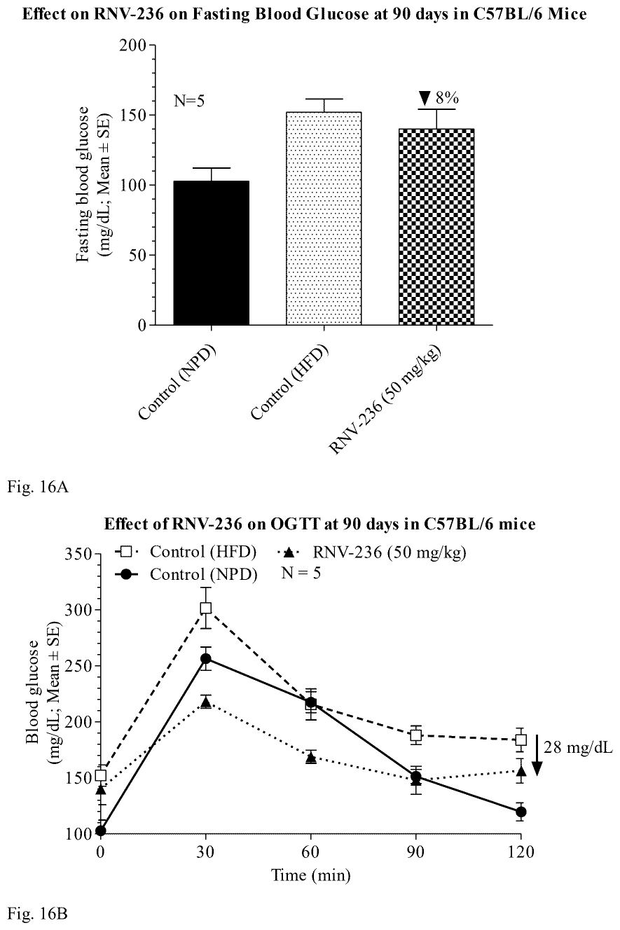

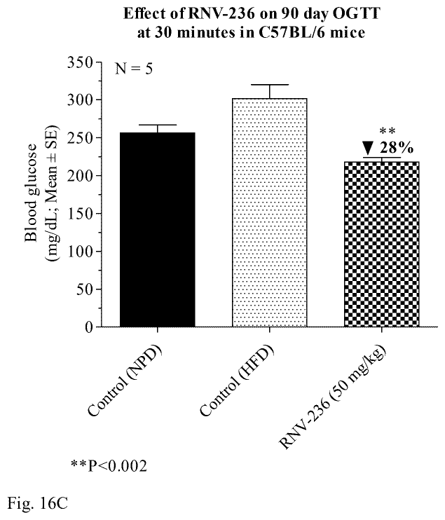

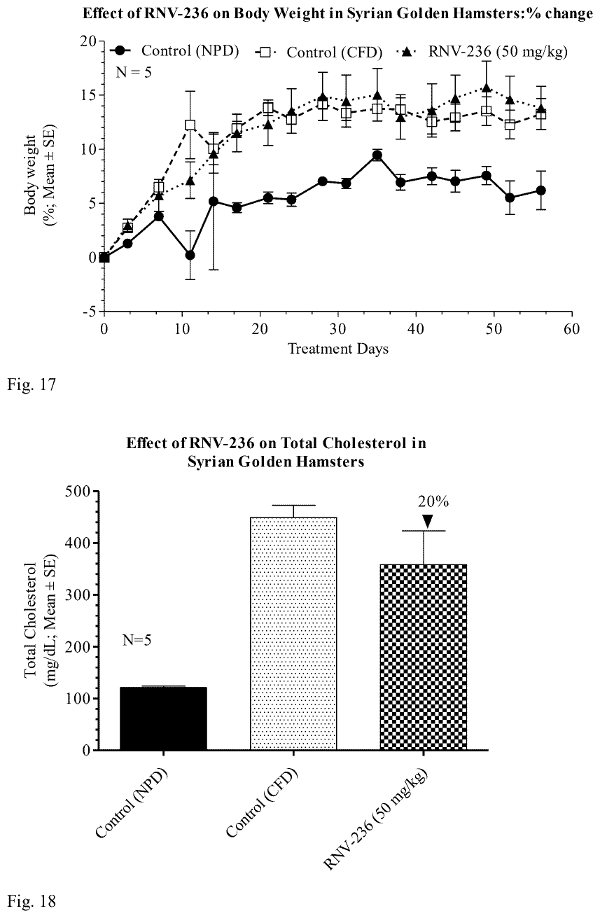

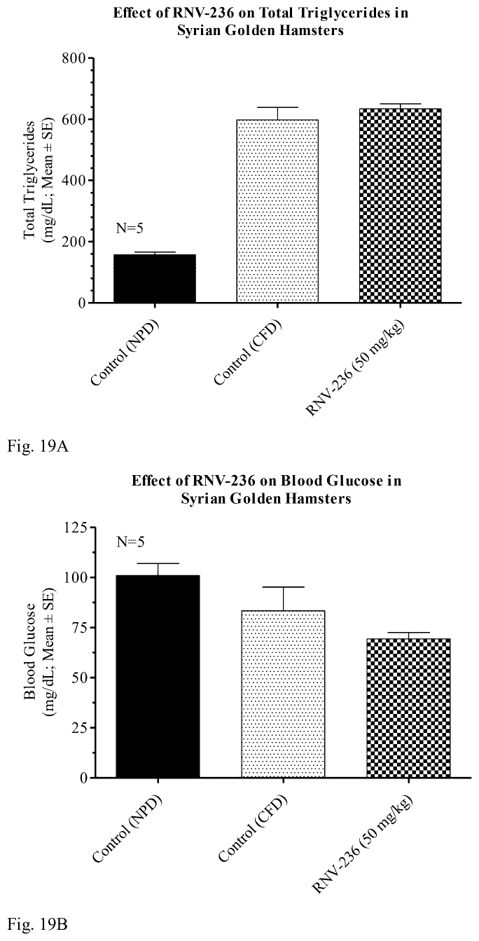

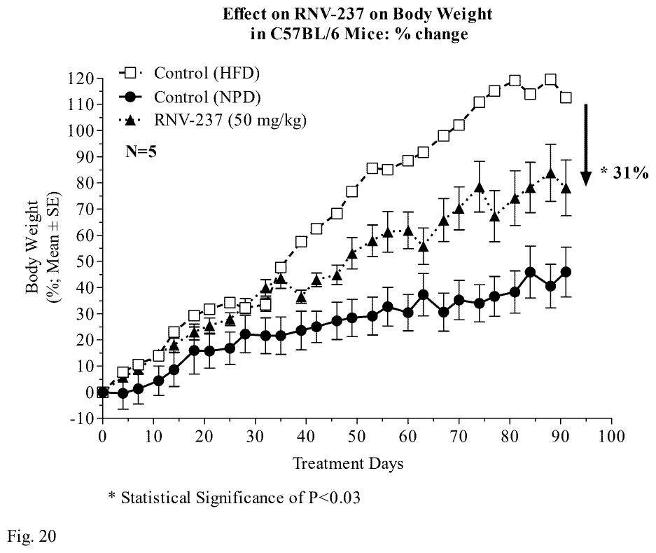

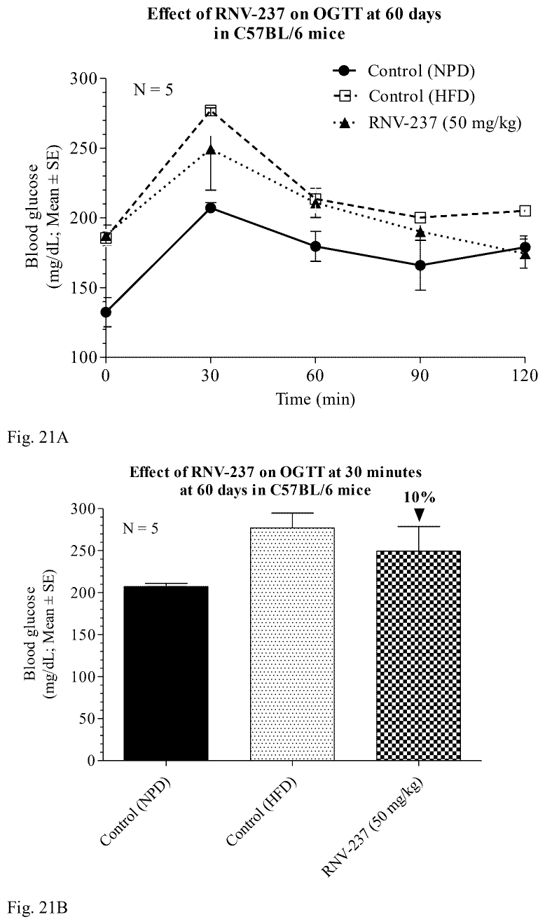

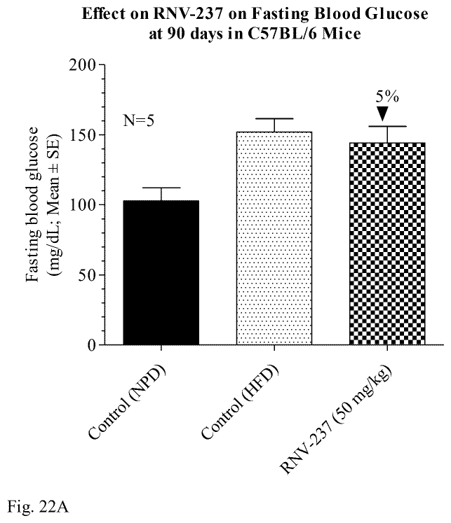

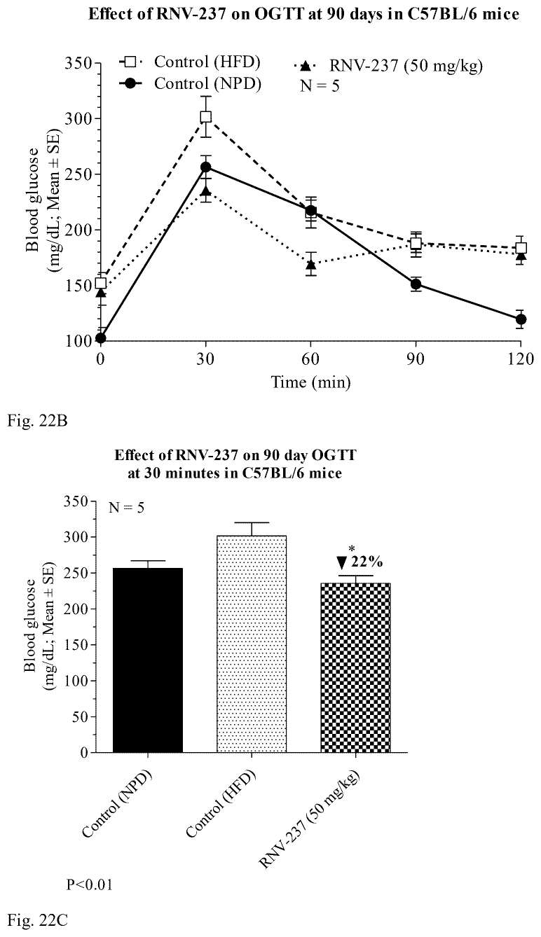

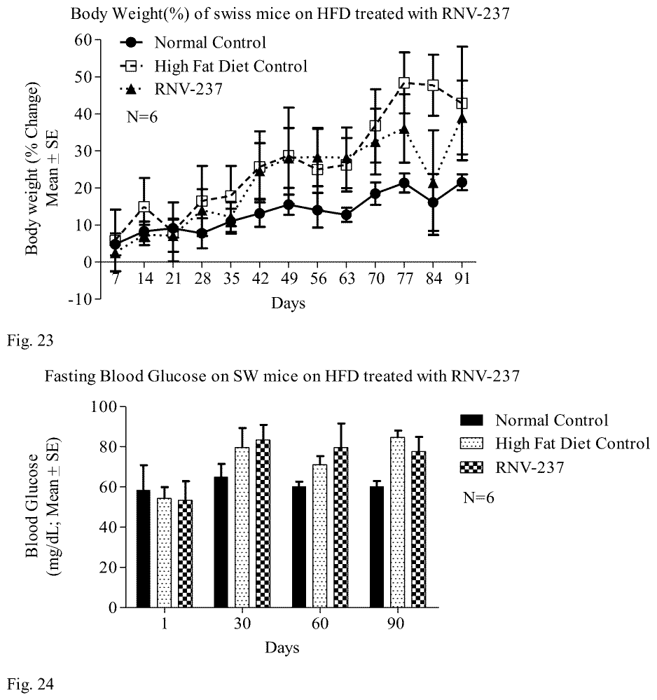

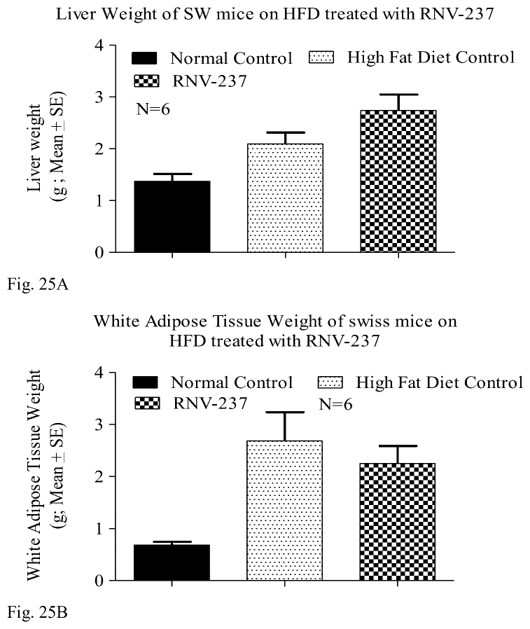

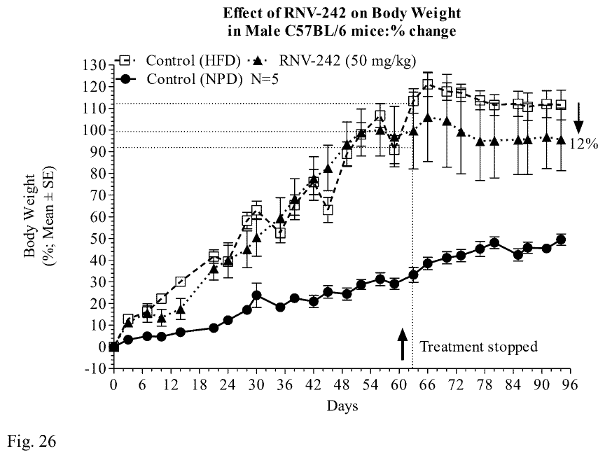

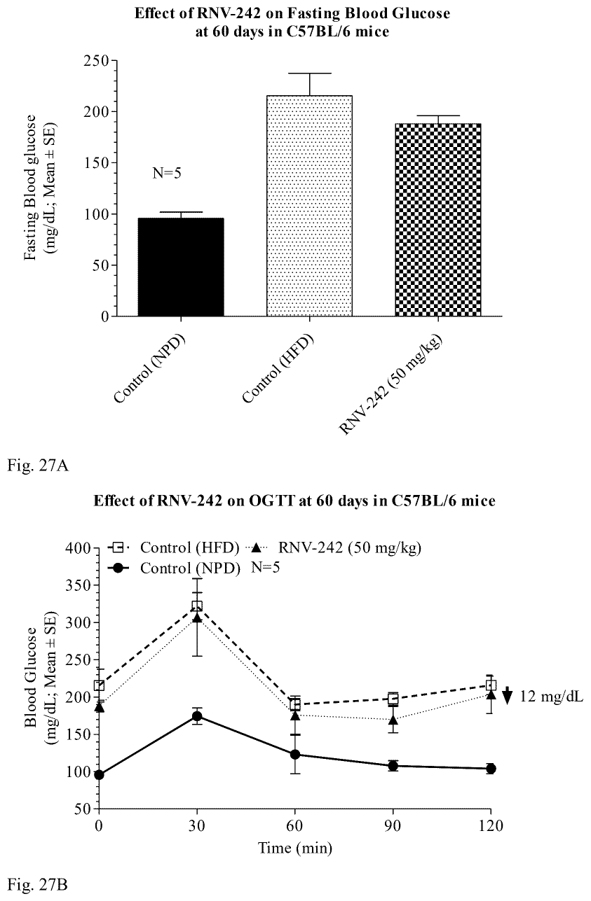

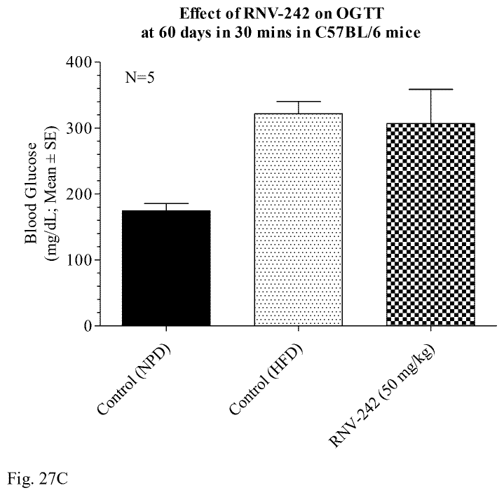

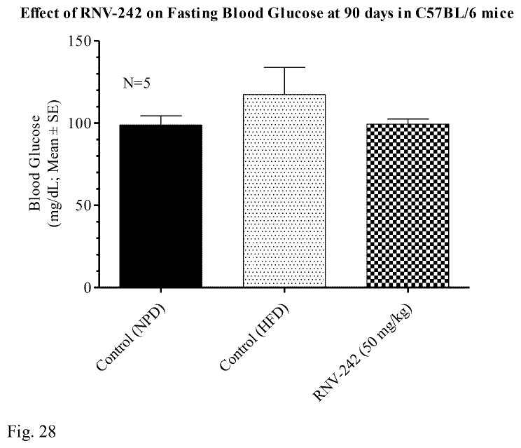

According to another feature of this present invention, there is provided a process for the preparation of the compound represented by the formula I, wherein all symbols are as defined as earlier, as shown in scheme-I (see FIG. 3).

Vanillin reacts with aromatic or heterocyclic acid chlorides which might be saturated or unsaturated in the presence any weak base to yield substituted vanillin. This further reacts with substituted aromatic hydrazines to yield Vanillin Hydrazones.

Embodiments of the invention include, for example, the following:

A compound formula I (see FIG. 1)

A compound of formula I, according to Claim 1, the group represented as X is derived from substituted or unsubstituted pyridyl or aryl acid chloride.

A compound of formula I, according to Claim 1, the groups represented as R1 to R8 are selected from Hydrogen, Halogens, hydroxyl, alkoxy, straight chain or branched alkyl, nitro, cyano and amino groups.

A compound of formula I, according to Claim 1, Pharmaceutically acceptable salts forming part of this invention include base addition salts such as alkali metal salts like Li, Na, and K salts, alkaline earth metal salts like Ca and Mg salts, salts of organic bases such as lysine, arginine, guanidine, diethanolamine, chlorine and the like, ammonium or substituted ammonium salts. Salts may include acid addition salts which are sulphates, nitrates, phosphates, perchlorates, borates, hydrohalides, acetates, tartarates, maleates, citrates, succinates, palmoates, methanesulphonates, benzoates, ascorbates, glycerophosphates, ketoglutarates and the like. Pharmaceutically acceptable solvates may be hydrates or comprising other solvents of crystallization such as alcohols.

A compound of formula I, according to Claim 1, their derivatives, their analogs, their tautomeric forms, their stereoisomers, their polymorphs, their pharmaceutically acceptable salts, their pharmaceutically acceptable solvates, novel intermediates and pharmaceutical composites containing them, wherein Part A, the vanillin ring has substituents R1, R2 and R3 which can be selected from Hydrogen, Halogens, hydroxyl, alkoxy, straight chain or branched alkyl, nitro, cyano and amino groups, and X, which can be selected from substituted or unsubstituted pyridyl or aryl acid chloride, and Part B having substituents R4, R5, R6, R7 and R8 which are selected from Hydrogen, Halogens, hydroxyl, alkoxy, straight chain or branched alkyl, nitro, cyano and amino groups.

A compound from claim 1 which is selected from the group consisting of

Nicotinic acid 4-[(2,4-dinitro-phenyl)-hydrazonomethyl]-2-methoxy-phenyl ester (RNV 203)

Benzoic acid-4-[(4-cyano phenyl)-hydrazonomethyl]-2-methoxy-phenyl ester (RNV-230)

3-phenyl-acrylic acid 4-formyl-2-methoxy-phenyl ester (RNV-233)

Nicotinic acid 4-[(4-cyano-phenyl)-hydrazonomethyl]-2-methoxy-phenyl ester (RNV-235)

Benzoic acid 2-bromo-4-[(2,4-dinitro-phenyl)-hydrazonomethyl]-6-methoxy-phenyl ester (RNV 236)

Benzoic acid 4-[(2,4-dinitro-phenyl)-hydrazonomethyl]-2-methoxy-phenyl ester (RNV-237)

Benzoic acid 2-bromo-4-hydrazonomethyl-6-methoxy-phenyl ester (RNV-242)

Toluene-4-sulfonic acid-4[(2,4-dinitro-phenyl)-hydrazonomethyl]-2-methoxy-phenyl ester (RNV-240)

Toluene-4-sulfonic acid-2-bromo-4[(2,4-dinitro-phenyl)-hydrazonomethyl]-6-methoxy-phenyl ester (RNV-369)

Toluene-4-sulfonic acid-4[(hexadecanoyl-hydrazonomethyl]-2-methoxy-phenyl ester (RNV-370)

Preferred salts for the compounds listed above are hydrochloride, hydrobromide, sodium, potassium or magnesium.

A process for the preparation of the above said novel compounds, their analogs, their tautomeric forms, their stereoisomers, their polymorphs, their pharmaceutically acceptable salts, their pharmaceutically acceptable solvates, novel intermediates and pharmaceutical composites containing them. Tautomeric forms are isomeric forms which exists in a state of equilibrium capable of reacting according to either form. Stereoisomers include configurational isomers, such as cis- and trans double bonds, as well as optically active isomers having different spatial arrangements of their atoms. Polymorphs are molecules which can crystallize in two or more forms. Solvates are molecular or ionic complexes of molecules or ions of solvent with those of a solute. The amino acid derivatives are included, but not limited to naturally occurring amino acids. Analogs include those compounds which differ by substitution of an oxygen, sulphur, nitrogen or carbon atom in place of such an atom. Analogs also include atoms of the same family of the Periodic Table, such as F, Cl, Br and I. Derivatives include compounds resulting from routine functionalizing of atoms, such as, derivatives found by protecting amino or carboxyl groups by carboxylation or esterification, respectively.

A process for the preparation of the compound represented by the formula I, wherein all symbols are as defined as earlier, as shown in scheme I (see FIG. 3)

Vanillin reacts with aromatic or heterocyclic acid chlorides which might be saturated or unsaturated in the presence any weak base to yield substituted vanillin. This further reacts with substituted aromatic hydrazines to yield Vanillin Hydrazones.

A method for reducing glucose in plasma comprising administering an effective amount of a compound of formula I as defined in claim 1 to a patient in need thereof

A method for reducing free fatty acid in plasma comprising administering an effective amount of a compound of formula I as defined in claim 1 to a patient in need thereof

A method for reducing cholesterol in plasma comprising administering an effective amount of a compound of formula I as defined in claim 1 to a patient in need thereof

A method for reducing triglyceride levels in plasma comprising administering an effective amount of a compound of formula I as defined in claim 1 to a patient in need thereof

A method for treating obesity comprising administering an effective amount of a compound of formula I as defined in claim 1 to a patient in need thereof

A method for treating autoimmune diseases comprising administering an effective amount of a compound of formula I as defined in claim 1 to a patient in need thereof

A method for treating inflammation comprising administering an effective amount of a compound of formula I as defined in claim 1 to a patient in need thereof

A method for treating immunological disease comprising administering an effective amount of a compound of formula I as defined in claim 1 to a patient in need thereof

A method according to claim 15, wherein the autoimmune disease is multiple sclerosis

A method according to claim 15, wherein the autoimmune disease is rheumatoid arthritis

A method according to claim 16, wherein the inflammation is mediated by cyclooxygenase

A method according to claim 17, wherein the immunological diseases is mediated by cytokines

A method for treating a disorder associated with insulin resistance comprising administering an effective amount of a compound of formula I as defined in claim 1 to a patient in need thereof

1. A compound of Formula I 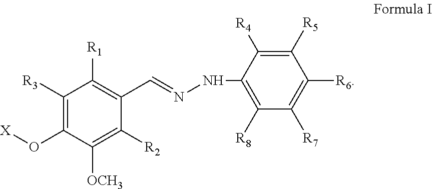
wherein X is derived from substituted or unsubstituted pyridyl or aryl acid chloride, and R1 to R8 are selected from Hydrogen, Halogens, hydroxyl, alkoxy, straight chain or branched alkyl, nitro, cyano and amino groups, and salts, derivatives, their analogs, their tautomeric forms thereof.

2. The compound of claim 1, wherein the salts thereof are pharmaceutically acceptable salts that include base addition salts such as alkali metal salts like Li, Na, and K salts, alkaline earth metal salts like Ca and Mg salts, salts of organic bases such as lysine, arginine, guanidine, diethanolamine, chlorine, ammonium and substituted ammonium salts.

3. The compound of claim 1, wherein in the vanillin ring has substituents R1, R2 and R3 which can be selected from Hydrogen, Halogens, hydroxyl, alkoxy, straight chain or branched alkyl, nitro, cyano and amino groups, and X, which can be selected from substituted or unsubstituted pyridyl or aryl acid chloride, and the other ring has substituents R4, R5, R6, R7 and R8 which are selected from Hydrogen, Halogens, hydroxyl, alkoxy, straight chain or branched alkyl, nitro, cyano and amino groups.

4. The compound of claim 1, selected from the group consisting of
Nicotinic acid 4-[(2,4-dinitro-phenyl)-hydrazonomethyl]-2-methoxy-phenyl ester (RNV 203),
Benzoic acid-4-[(4-cyano phenyl)-hydrazonomethyl]-2-methoxy-phenyl ester (RNV-230),
3-phenyl-acrylic acid 4-formyl-2-methoxy-phenyl ester (RNV-233),
Nicotinic acid 4-[(4-cyano-phenyl)-hydrazonomethyl]-2-methoxy-phenyl ester (RNV-235),
Benzoic acid 2-bromo-4-[(2,4-dinitro-phenyl)-hydrazonomethyl]-6-methoxy-phenyl ester (RNV 236),
Benzoic acid 4-[(2,4-dinitro-phenyl)-hydrazonomethyl]-2-methoxy-phenyl ester (RNV-237),
Benzoic acid 2-bromo-4-hydrazonomethyl-6-methoxy-phenyl ester (RNV-242),
Toluene-4-sulfonic acid-4[(2,4-dinitro-phenyl)-hydrazonomethyl]-2-methoxy-phenyl ester (RNV-240),
Toluene-4-sulfonic acid-2-bromo-4[(2,4-dinitro-phenyl)-hydrazonomethyl]-6-methoxy-phenyl ester (RNV-369), and
Toluene-4-sulfonic acid-4[(hexadecanoyl-hydrazonomethyl]-2-methoxy-phenyl ester (RNV-370).

5. The compound of claim 1, wherein the salts for the compounds are hydrochloride, hydrobromide, sodium, potassium or magnesium.

6. A process for the preparation of the compound of claim 1 wherein vanillin reacts with aromatic or heterocyclic acid chlorides in the presence any weak base to yield substituted vanillin which further reacts with substituted aromatic hydrazines to yield Vanillin Hydrazones.

10. A method for treating a disease or affecting a physiological change in a subject comprising administering to the subject a pharmaceutically effective dose of the compound of claim 1, where the change comprises at least one change selected from the group consisting of: (i) reducing glucose in plasma; (ii) reducing free fatty acid in plasma; (iii) reducing cholesterol in plasma; and (iv) reducing triglyceride levels in plasma.

11. The method of claim 10 wherein the disease is selected from the group consisting of: obesity, an autoimmune disease, an inflammatory disease, and an immunological disease.

12. The method of claim 11 wherein the autoimmune disease is multiple sclerosis.

13. The method of claim 11 wherein the autoimmune disease is rheumatoid arthritis.

14. The method of claim 11 wherein the autoimmune disease is wherein the inflammatory disease is mediated by cyclooxygenase.

15. The method of claim 11 wherein the inflammatory disease wherein the immunological disease is mediated by cytokines.

16. The method of claim 10 wherein the disease is associated with insulin resistance.
